(19) United States
(12) Patent Application Publication
(10) Pub. No.: US 2003/0166178 A1
(43) Pub. Date: Sep. 4, 2003

(54) Process for the production of enantiomer-enriched alpha-substituted carboxylic acids

(75) Inventors: Oliver May, Frankfurt (DE); Christoph Syldatk, Stuttgart (DE); Oliver Vielhauer, Stuttgart (DE); Markus Werner, Weinsberg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf, DE

(21) Appl. No.: 10/105,251

(22) Filed: Mar. 26, 2002

(30) Foreign Application Priority Data
Mar. 26, 2001 (DE) 101 15 000.8

(51) Int. Cl. C12P 7/40

(52) U.S. Cl. 435/136

(57) ABSTRACT

The present invention provides enzymatic processes for the production of enantiomer-enriched u-substituted carboxylic acids.

### CROSS-REFERENCE TO RELATED APPLICATIONS

The present application claims priority to German Application No. DE 101 15 000.8, filed on Mar. 26, 2001, which is hereby incorporated by reference in its entirety.

### BACKGROUND OF THE INVENTION

1. Field of the Invention

The present invention relates to enzymatic processes for the production of enantiomer-enriched α-substituted carboxylic acids.

2. Discussion of the Background

In organic synthesis, α-substituted carboxylic acids serve as important intermediates for the production of several classes of compounds. However, the synthesis of some of these classes, such as amino acids and diols, require the α-substituted carboxylic acids to be present to a high optical purity, particularly when the α-substituted carboxylic acid is to be used for the production of bioactive substances or catalysts.

One such process is described in WO0058449 (and references cited therein), which describes an enzymatic reaction of hydantoins to yield enantiomer-enriched amino acids. However, processes for the complete conversion of hydantoin analogues to optically enriched α-substituted carboxylic acids are not known. Therefore, there is a critical need for methods of producing enantiomer-enriched populations of α-substituted carboxylic acids from racemic mixtures of hydantoin analogues.

α-Substituted carboxylic acids, which constitute the subject matter of the present invention may be obtained by processes known in the art. For example, the optical antipodes of the compounds under consideration may be obtained by conventional racemate resolution of the racemic mixtures or by chromatographing them on chiral phases. The racemic mixtures themselves are sometimes cheap compounds, while some enantiomers of the α-substituted carboxylic acids may also be obtained from the chiral pool.

### SUMMARY OF THE INVENTION

Therefore, it is an object of the present invention to provide a process for the production of enantiomer-enriched α-substituted carboxylic acids.

Another object of the present invention is to provide a process capable of providing a wide range of compounds at the highest possible optical purities and at the lowest possible cost. Further, it is an object of the present invention to provide such a process that is industrially robust.

Another object of the present invention is a process for the production of enantiomer enriched α-substituted carboxylic acids by transforming compounds of formula (I)

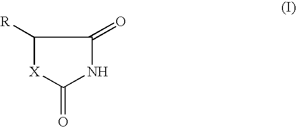

wherein, X is O, S, or CH₂, and R is an enzyme-reactive organic residue with enzymatic ring cleavage by a hydantoinase to form intermediate product of formula (II);

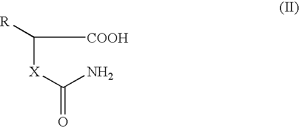

wherein, X is O, S, CH₂, and R is an enzyme-reactive organic residue. Subsequently, the compound of formula (II) is converted by a carbamoylase to yield the desired enantiomer-enriched α-substituted carboxylic acid.

In another object of the present invention a process is provided in which enantiomer-enriched α-substituted carboxylic acids are produced by a reaction with a carbamoylase with compounds of formula (II)

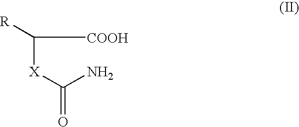

wherein X is O, S, or CH₂, and R is an enzyme-reactive organic residue.

Another object of the present invention is to provide a process in which a racemase is also used in addition to a hydantoinase and/or a carbamoylase to yield enantiomer-enriched α-substituted carboxylic acids.

Another object of the invention relates to the use of the enantiomer-enriched α-substituted carboxylic acids as a substrate in organic synthesis, inter alia for the synthesis of catalysts, or for the production of bioactive compounds.

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in biochemistry, cellular biology, and organic chemistry.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Enantiomer-enriched α-substituted carboxylic acids according to the invention are important intermediates for organic synthesis, but they may also be used to obtain a range of other important optically highly pure classes of compounds, such as amino acids, diols etc., which may be used in organic synthesis for the production of bioactive substances or as catalysts.

In the process for the production of enantiomer-enriched α-substituted carboxylic acids by transforming compounds of formula (I)

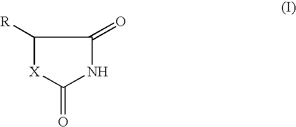

wherein, X is O, S, or CH₂, and R is an enzyme-reactive organic residue reacts with enzymatic ring cleavage by a hydantoinase to form intermediate product of formula (II)

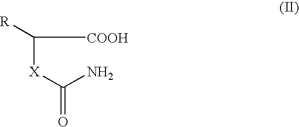

wherein, X is O, S, or CH₂, and R is an enzyme-reactive organic residue reacts. Subsequently, the intermediate of formula (II) is converted by a carbamoylase to yield the desired enantiomer-enriched α-substituted carboxylic acid.

In another embodiment, the process involves producing enantiomer-enriched α-substituted carboxylic acids by a reaction with a carbamoylase and compounds of formula (II)

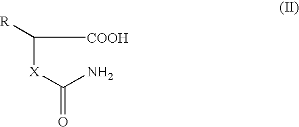

wherein X is O, S, or CH₂, and R is an enzyme-reactive organic residue.

“Optically enriched” or “enantiomer-enriched” compounds within the scope of the present invention is understood to mean the presence of an optical antipode mixed with the other antipodes in a concentration of >50 mole %. Further, within the scope of the present invention are compounds that relate to both D- and L- optical isomers.

In principal, suitable R groups may be any organic residue that permits reaction of the compounds of the formulae (I) or (II) in the enzyme systems of the present invention. The determination of suitable R groups is readily ascertainable by the skilled artisan through routine experimentation. Preferred R groups for use in the present invention include (C₁-C₈)-alkyl, (C₁-C₈)-alkoxy, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl, (C₁-C₈)-acyl, and (C₁-C₈)-acyloxy. More preferred R groups may be those which match the corresponding α-residues of proteinogenic or natural amino acids. However, the R group may correspond to the α-residues of non-proteinogenic or unnatural amino acids. Natural (or proteinogenic) α-amino acids are described in Bayer-Walter, Lehrbuch der organischen Chemie, S. Hirzel Verlag, Stuttgart, 22nd edition, 1991, pp. 822 et seq.. Furthermore, preferred examples of unnatural or non-proteinogenic α-amino acids are described in DE 19903268.8. In addition, the side chain residues may be derived from the α-amino acids presented therein.

(C₁-C₈)-Alkyl groups should be taken to mean methyl, ethyl, n-propyl, isopropyl, n-butyl, isobutyl, sec-butyl, tert-butyl, pentyl, hexyl, heptyl or octyl, including all bond isomers thereof. The (C₁-C₈)-alkoxy group corresponds to a (C₁-C₈)-alkyl group with the proviso that the latter is attached to the molecule via an oxygen atom. (C₂-C₈)-Alkoxyalkyl is intended to mean groups in which the alkyl chain is interrupted by at least one oxygen function, where two oxygen atoms may not be joined together. The number of carbon atoms indicates the total number of carbon atoms present in the substituent group.

The aforementioned substituents may be mono- or polysubstituted with halogens and/or residues containing N, O, P, S or Si atoms. These are in particular alkyl residues of the above-stated type which contain one or more of these heteroatoms in their chain or which are attached to the molecule via one of these heteroatoms.

(C₃-C₈)-Cycloalkyl is a cyclopropyl, cyclobutyl, cyclopentyl, cyclohexyl or cycloheptyl residues etc.. These may be substituted with one or more halogens and/or residues containing N, O, P, S, Si atoms and/or comprise N, O, P, S atoms in the ring, for example 1-,2-,3-,4-piperidyl, 1-,2-,3-pyrrolidinyl, 2-,3-tetrahydrofuryl, 2-,3-,4-morpholinyl.

A (C₃-C₈)-cycloalkyl-(C₁-C₈)-alkyl designates a cycloalkyl residue as described above which is attached to the molecule via an alkyl residue as stated above.

For the purposes of the invention, (C₁-C₈)-acyloxy means an alkyl residue having at most 8 C atoms as described above which is attached to the molecule via a COO function.

For the purposes of the invention, (C₁-C₈)-acyl means an alkyl residue having at most 8 C atoms as described above which is attached to the molecule via a CO function.

A (C₆-C₁₈)-aryl residue is an aromatic residue having 6 to 18 C atoms. In particular, these compounds include those such as phenyl, naphthyl, anthryl, phenanthryl, biphenyl residues or systems of the above-described type fused to the molecule in question, such as indenyl systems, which may optionally be substituted with (C-C₈)-alkyl, (C₁-C₈)-alkoxy, N(C₁-C₈)-alkyl, (C₁-C₈)-acyl, (C₁-C₈)-acyloxy.

A (C₇-C₁₉)-aralkyl residue is a (C₁-C₈)-aryl residue attached to the molecule via a (C₆-C₁₈)-alkyl residue.

For the purposes of the invention, a (C₃-C₁₈)-heteroaryl residue means a five-, six- or seven-membered aromatic ring system comprising 3 to 18 C atoms which comprises heteroatoms, such as nitrogen, oxygen or sulfur in the ring. Such heteroaromatic compounds are in particular taken to be residues such as 1-,2-,3-furyl, such as 1-,2-,3-pyrrolyl, 1-,2-,3-thienyl, 2-,3-,4-pyridyl, 2-,3-,4-,5-,6-,7-indolyl, 3-,4-,5-pyrazolyl, 2-,4-,5- 5 imidazolyl, acridinyl, quinolinyl, phenanthridinyl, 2-,4-, 5-,6-pyrimidinyl.

A (C₄-C₁₉)-heteroalkyl is a heteroaromatic system corresponding to the (C₇-C₁₉)-aralkyl residue.

Suitable halogens (Hal) which may be employed in the above substituent groups are fluorine, chlorine, bromine and iodine.

In the process of the present invention, a racemase may also be used to yield enantiomer-enriched α-substituted carboxylic acids.

In this process, the racemase is contacted with compounds (I) and/or (II) is treated with a racemase. As shown in Scheme (I), a consequence of this treatment is that a racemic starting product may almost completely give rise to an enantiomer of the α-substituted carboxylic acids according to the present invention.

In another embodiment of the invention, racemisation proceed by other means, in particular by chemical processes may be employed. Suitable racemisation processes and racemases are described, for example, in EP0542098, DE10050124.9, DE10050123.0, DE19935268 and DE19529211.1. Preferred racemases for use in the present invention include a hydantoin racemase, a carbamoylamino acid racemase, a or N-acetylamino acid racemase.

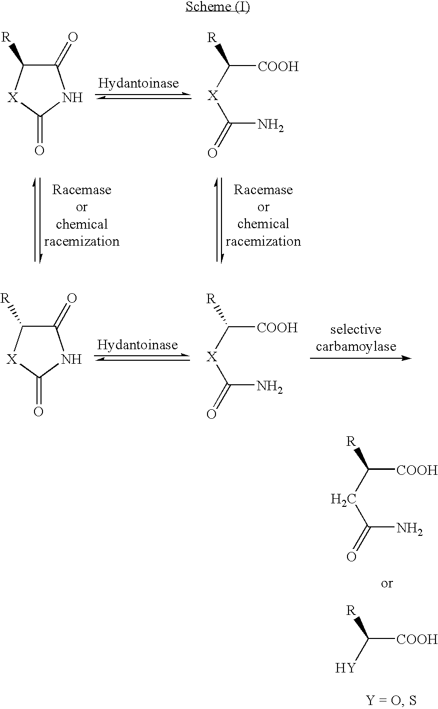

A preferred embodiment of the present invention is a process in which the enzymes used are provided in recombinant manner by expression from host organisms (thesis by Martin Hils: Mutanten der D-Carbamoylase zur Bildung aktiven Enzyms bei Expression des Gens in Escherichia coli and Analyse eines Genclusters fur die Enzyme des Hydantoin-Abbaus aus Agrobacterium sp IP I-671; Verlag Ulrich E. Grauer, Stuttgart 1998). Accordingly, the reaction may be performed with the assistance of a host organism, which is capable of expressing the appropriate enzymes. Preferably, all the enzymes to be used to achieve this object are expressed by a single host organism (whole cell catalyst WO0058449).

The form of the host organism expressing the hydantoinase, carbamoylase, and/or racemase to be used in the reactions of the present invention is not particularly limiting. For example, the host organism may be undisrupted, partially disrupted, or entirely disrupted. Further, the host organism may be used solely as an expression source from which the aforementioned enzymes may be purified to virtual homogeneity. The essentially homogenous enzymes may then be used to achieve the objects of the present invention.

Examples of host organisms suitable for use with the present invention include the E. coli strains NM 522, JM109, RR1, DH5α, TOP 10, and HB101.

In principle, any enzymes which may be considered by the person skilled in the art for the purpose according to the invention may be used for the reaction. Descriptions of the preferable hydantoinase, carbamoylase, and racemase enzymes for use in the present invention may be found in “Enzyme Catalysis in Organic Synthesis”, (eds.: Drauz, Waldmann, VCH, 1^{st} ed., 1995).

Hydantoinases from the organism Bacillus sp., Agrobacterium sp. or Arthrobacter sp. are preferable to achieve the objects of the present invention. A preferred hydantoinase is the commercially available hydantoinase 1 from Roche Diagnostics GmbH or the hydantoinase from Arthrobacter aurescens DSM3747 (SEQ ID NO: 4) or Arthrobacter aurescens DSM3745 (SEQ ID NO: 3). A preferred source of the hydantoinase is Bacillus thermoglucosidasius.

The carbamoylase to be used in the present invention may be obtained from Agrobacterium radiobacter IP I-671 (SEQ ID NO: 1) or Arthrobacter crystallopoietes DSM 20117 (SEQ ID NO: 2) are preferably used as the carbamoylase. Agrobacterium radiobacter IP 1-671 and Arthrobacter crystallopoietes DSM 20117 are commercially available.

Preferable racemases for use in the present invention include a hydantoin racemase, a carbamoylamino acid racemase, or a N-acetylamino acid racemase (DE1 0050124.9, DE10050123.0, U.S. Ser. No. 09/407062, WO0058449).

In one embodiment of the present invention, the reaction is preferably performed in an enzyme membrane reactor (DE 199 10 691.6).

The present invention also provides a use of the enantiomer-enriched α-substituted carboxylic acids as a substrate in organic synthesis, inter alia for the synthesis of catalysts, or for the production of bioactive compounds.

The compounds of the formula (I) to be used in the reaction according to the invention may be produced using methods known in the art. The oxa- or thia- analogous hydantoin structures may accordingly be obtained from the corresponding racemic α-hydroxy or α-mercaptocarboxylic acids by reaction with phosgene or methoxy- or benzyloxycarbonyl chloride (by analogy with H. Leuchs. Ber. 39, 857 (1906)). An alternative method which could be employed is the condensation of 2,4-dioxothiazolidines or 2,4-dioxooxazolidines with aldehydes and subsequent reduction of the double bond by analogy with hydantoin syntheses (Beyer-Walter, Lehrbuch der organischen Chemie, S. Hirzel Verlag, Stuttgart, 22nd edition, 1991, p. 744). An additional overview of suitable methods of producing compounds of formula (I) are provided in the thesis by T. Waniek (University of Stuttgart 1999).

As previously stated, the enzymes of the present invention may be used together or in succession. Additionally, these enzymes may be in free form as homogeneously purified compounds or as enzymes produced by recombinant means. Further, the enzymes may also be used as a constituent of a guest organism (whole cell catalyst as in U.S. Ser. No. 09/407062, WO0058449) or in conjunction with the disrupted cell mass of the host organism. It may also be possible to use the enzymes in an immobilised form (Bhavender P. Sharma, Lorraine F. Bailey and Ralph A. Messing, “Immobilisierte Biomaterialiem—Techniken and Anwendungen”, Angew. Chem. 1982, 94, 836-852).

Enzyme immobilisation may be achieved, for example, by freeze-drying (Dordick et al. J. Am. Chem. Soc. 1994, 116, 5009-5010; Okahata et al. Tetrahedron Lett. 1997, 38, 1971-1974; Adlercreutz et al. Biocatalysis 1992, 6, 291-305). In a preferred method, freeze-drying is performed in the presence of surface-active substances, such as Aerosol OT, polyvinylpyrrolidone, polyethylene glycol (PEG), or Brij 52 (diethylene glycol monocetyl ether; Goto et al., Biotechnol. Techniques 1997, 11, 375-378).

Having generally described this invention, a further understanding can be obtained by reference to certain specific examples, which are provided herein for purposes of illustration only, and are not intended to be limiting unless otherwise specified.

### EXAMPLES

### Example 1

Regio- and enantio-specific hydrolysis of BnBHA with D-carbamoylases (hydantoinase reaction, c.f. thesis of T. Waniek, Stuttgart 1999).

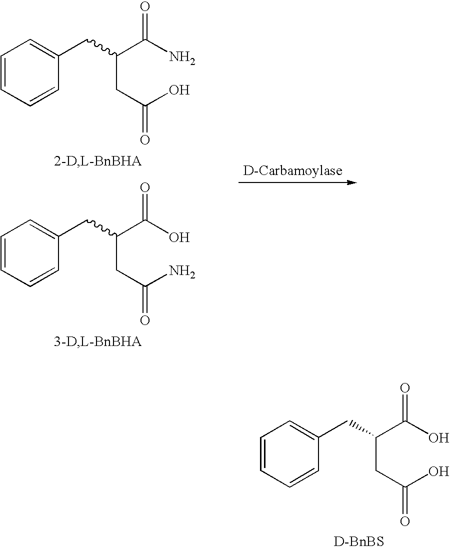

A mixture of D,L-2-BnBHA and D,L-3-BnBHA (44:56, HPLC) was dissolved at a concentration of 2 g/l (9.6 mM) in 200 μl of 0.1 M potassium phosphate buffer, pH 7.0, and combined with 80 μl of enzyme solution. Incubation was performed in a Thermomixer at 37° C. for the D-carbamoylase from Agrobacterium radiobacter IP I-671 and at 30° C. for the D-carbamoylase from Arthrobacter crystallopoietes DSM 20117. The reaction was terminated by combining 280 μl of reaction solution with 100 μl of 10% phosphoric acid. The reaction batch was centrifuged and the supernatant diluted 1:10 with HPLC mobile solvent and analysed.

After a reaction time of 10 min, RP₁₈HPLC revealed 3-BnBHA conversion of 50%, while 2-BnBHA was not converted.

Chiral HPLC analysis (cyclodextrin phase) revealed that the resultant D-BnBS was enantiomerically pure.

Specific enzyme activity was determined at least 3.2 U/mg.

### Example 2

Enantio-specific hydrolysis of CPhM with a D-carbamoylase (hydantoinase reaction, c.f. thesis of T. Waniek, Stuttgart, 1999).

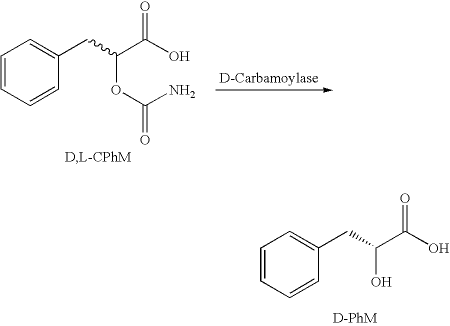

The substrates were tested both with the D-carbamoylase from Agrobacterium sp. IP I-671 and with that from Arthrobacter crystallopoietes DSM 20117. Where the reaction conditions with the D-carbamoylase vary for Arthrobacter, the different values are given in brackets.

The D,L-CPhM was dissolved at a concentration of 2 g/l in 400 gl of 0.1 M potassium phosphate buffer, pH 7.0, and combined with 200 μl of enzyme solution. Incubation was performed in a Thermomixer at 37° C. (30° C.). The reaction was terminated by combining 600 μl of reaction solution with 200 μl of 10% phosphoric acid. The reaction batch was centrifuged and the supernatant diluted 1:10 with HPLC mobile solvent and analysed.

After a reaction time of 60 min (10 min), RP₁₈ HPLC revealed conversion of 33% (2.8%).

Chiral HPLC analysis (cyclodextrin phase) revealed that only D-PhM had been obtained as the product of the enzymatic reaction.

Specific enzyme activity was determined at 0.2 U/mg (0.05 U/mg).

### Example 3

Single-Vessel Reaction

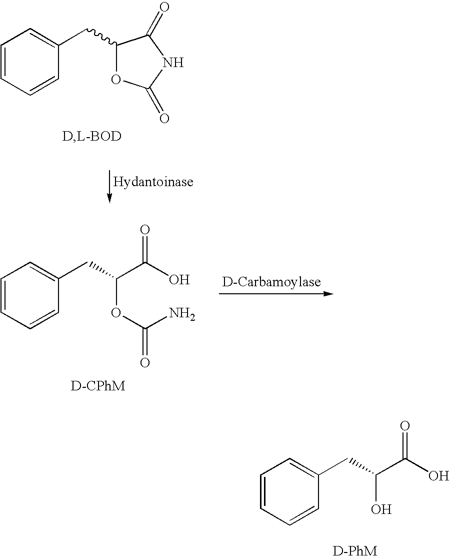

D,L-BOD was dissolved at a concentration of 2 g/l in 1 ml of 0.1 M potassium phosphate buffer, pH 7.0, and combined with 200 μl of L-hydantoinase from Arthrobacter aurescens DSM 3747 immobilised on Eupergit and with 200 μl of D-carbamoylase solution (Agrobacterium sp. IP I-671). Incubation was performed in a Thermomixer at 37° C. The reaction was terminated by combining the immobilisate with 200 μl of 10% phosphoric acid, centrifuging the mixture and diluting the supernatant 1:10 in HPLC mobile solvent and performing analysis.

After a reaction time of 24 h, RP₁₈ HPLC revealed that both CPhM (˜20%) and PhM (˜5%) had been obtained.

Numerous modifications and variations on the present invention are possible in light of the above teachings. It is, therefore, to be understood that within the scope of the accompanying claims, the invention may be practiced otherwise than as specifically described herein.

1. A process for the production of an enantiomer-enriched α-substituted carboxylic acid comprising
a) contacting a compound of formula (I) with a hydantoinase 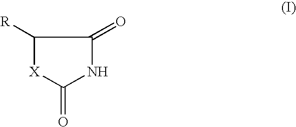
wherein, X is O, S, or CH₂, and R is an enzyme-reactive organic residue, to form a product of formula (II) 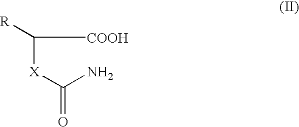
wherein X and R are as defined above; and
b) contacting the product of formula (ii) with a carbamoylase:

2. The process according to claim 1, wherein R is selected from the group consisting of (C₁-C₈)-alkyl, (C₁-C₈)-alkoxy, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl, (C₁-C₈)-acyl, (C₁-C₈)-acyloxy, an α-residue of a natural amino acid, and an α-residue of an unnatural amino acid.

3. The process according to claim 1, wherein the contacting in step a) is performed in the presence of a host organism expressing at least one enzyme selected from the group consisting of the hydantoinase and the carbamoylase.

4. The process according to claim 1, further comprising contacting the compound of formula (I) with a racemase.

5. The process according to claim 4, wherein the racemase is selected from the group consisting of a hydantoin racemase, a carbamoylamino acid racemase, and a N-acetylamino acid racemase.

6. The process according to claim 4, wherein the contacting in step a) is performed in the presence of a host organism expressing at least one enzyme selected from the group consisting of the hydantoinase, the carbamoylase, and the racemase.

7. The process according to claim 1, further comprising contacting the compound of formula (II) with a racemase.

8. The process according to claim 7, wherein the racemase is selected from the group consisting of a hydantoin racemase, a carbamoylamino acid racemase, and a N-acetylamino acid racemase.

9. The process according to claim 7, wherein the contacting in step a) is performed in the presence of a host organism expressing at least one enzyme selected from the group consisting of the hydantoinase, the carbamoylase, and the racemase.

10. The process according to claim 1, wherein the hydantoinase is a Bacillus thermoglucosidasius hydantoinase.

11. The process according to claim 1, wherein the carbamoylase is a Agrobacterium radiobacter carbamoylase.

12. The process according to claim 1, which is performed in an enzyme membrane reactor.

13. A process of producing a catalyst or a bioactive compound, comprising converting the compound obtained by the process of claim 1 into a catalyst or a bioactive compound.

14. A process for the production of an enantiomer-enriched α-substituted carboxylic acid comprising contacting a carbamoylase with a compound of formula (II) 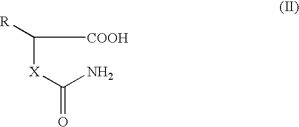
wherein X is O, S, or CH₂, and R is an enzyme-reactive organic residue.

15. The process according to claim 14, wherein R is selected from the group consisting of (C₁-C₈)-alkyl, (C₁-C₈)-alkoxy, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl, (C₁-C₈)-acyl, (C₁-C₈)-acyloxy, an (α-residue of a natural amino acid, and an α-residue of an unnatural amino acid.

16. The process according to claim 14, wherein the contacting in step a) is performed in the presence of a host organism expressing the carbamoylase.

17. The process according to claim 14, further comprising contacting the compound of formula (II) with a racemase.

18. The process according to claim 17, wherein the racemase is selected from the group consisting of a hydantoin racemase, a carbamoylamino acid racemase, and a N-acetylamino acid racemase.

19. The process according to claim 17, wherein the contacting in step a) is performed in the presence of a host organism expressing at least one enzyme selected from the group consisting of the carbamoylase and the racemase.

20. The process according to claim 17, wherein the carbamoylase is a Agrobacterium radiobacter carbamoylase.

21. The process according to claim 17, which is performed in an enzyme membrane reactor.

22. A process of producing a catalyst or a bioactive compound, comprising converting the compound obtained by the process of claim 17 into a catalyst or a bioactive compound.


SEQUENCE LISTING (4 sequences)

SEQ ID NO: 1 - protein, 304 aa, Agrobacterium radiobacter
MTRQMILAVGQQGPIARAETREQVIARLLDMLANAASRGVNFIVFPELAVTTFFPRWHLTDEAELDSFYETEMPGPLTRPLFEKAAELGIGFNFGYAELVVEGGVKRRFNTSILVDRSGKIIGKYRKVHLPGHKEYEAYRPFQHLEKRYFEPGDMGFPVYDVDAAKMGMFICNDRRWPEAWRVMGLKGAEIICGGYNTPTHNPAVPQHDHLTSFHHLLSMQAGSYQNGAWSAAAGKVGMEEGCMLLGHSCIVAPTGEIVALTTTLEDEVITATIDLDRCRELREHIFNFKAHRQPQHYGLIAEL

SEQ ID NO: 2 - protein, 315 aa, Arthrobacter crystallopoietes
LAKNLMLAVAQVGGIDSSESRPEVVARLIALLEEAASQGAELVVFPELTLTTFFPRTWFEEGDFEEYFDKSMPNDDVAPLFERAKDLGVGFYLGYAELTSDEKRYNTSILVNKHGDIVGKYRKMHLPGHADNREGLPNQHLEKKYFREGDLGFGVFDFHGVQVGMCLCNDRRWPEVYRSLALQGAELVVLGYNTPDFVPGWQEEPHAKMFTHLLSLQAGAYQNSVFVAAAGKSGFEDGHHMIGGSAVAAPSGEILAKAAGEGDEVVVVKADIDMGKPYKESVFDFAAHRRPDAYGIIAERKGRGAPLPVPFNVND

SEQ ID NO: 3 - protein, 458 aa, Arthrobacter aurescens
MFDVIVKNCRLVSSDGITEADILVKDGKVAAISADTSDVEASRTIDAGGKFVMPGVVDEHVHIIDMDLKNRYGRFELDSESAAVGGITTIIEMPITFPPTTTLDAFLEKKKQAGQRLKVDFALYGGGVPGNLPEIRKMHDAGAVGFKSMMAASVPGMFDAVSDGELFEIFQEIAACGSVIVVHAENETIIQALQKQIKAAGGKDMAAYEASQPVFQENEAIQRALLLQKEAGCRLIVLHVSNPDGVELIHQAQSEGQDVHCESGPQYLNITTDDAERIGPYMKVAPPVRSAEMNIRLWEQLENGLIDTLGSDHGGHPVEDKEPGWKDVWKAGNGALGLETSLPMMLTNGVNKGRLSLERLVEVMCEKPAKLFGIYPQKGTLQVGSDADLLILDLDIDTKVDASQFRSLHKYSPFDGMPVTGAPVLTMVRGTVVAEKGEVLVEQGFGQFVTRRNYEASK

SEQ ID NO: 4 - protein, 458 aa, Arthrobacter aurescens
MFDVIVKNCRLVSSDGITEADILVKDGKVAAISADTSDVEASRTIDAGGKFVMPGVVDEHVHIIDMDLKNRYGRFELDSESAAVGGITTIIEMPITFPPTTTLDAFLEKKKQAGQRLKVDFALYGGGVPGNLPEIRKMHDAGAVGFKSMMAASVPGMFDAVSDGELFEIFQEIAACGSVIVVHAENETIIQALQKQIKAAGGKDMAAYEASQPVFPENEAIQRALLLYKEAGCRLIVLHVSNPDGVELIHQAQSEGQDVHCESGPQYLNITTDDAERIGPYMKVAPPVRSAEMNIRLWEQLENGLIDTLGSDHGGHPVEDKEPGWKDVWKAGNGALGLETSLPMMLTNGVNKGRLSLERLVEVMCEKPAKLFGIYPQKGTLQVGSDADLLILDLDIDTKVDASQFRSLHKYSPFDGMPVTGAPVLTMVRGTVVAEKGEVLVEQGFGQFVTRRNYEASK